(19) United States
(12) Patent Application Publication
(10) Pub. No.: US 2013/0177610 A1
(43) Pub. Date: Jul. 11, 2013

(54) NANO-METALLIC ALLOY DELIVERY SYSTEM FOR TREATMENT OF INFECTED CELLS AND LEGIONS

(71) Applicant: UNIVERSITY OF NORTH CAROLINA AT GREENSBORO, Greensboro, NC (US)

(72) Inventor: Yousef Haik, Greensboro, NC (US)

(73) Assignee: UNIVERSITY OF NORTH CAROLINA AT GREENSBORO, Greensboro, NC (US)

(21) Appl. No.: 13/780,520

(22) Filed: Feb. 28, 2013

Related U.S. Application Data
(63) Continuation of application No. 13/299,791, filed on Nov. 18, 2011, now abandoned.
Application No. 13/299,791 is a division of application No. 12/512,726, filed on Jul. 30, 2009, now abandoned.
(60) Provisional application No. 61/085,375, filed on Jul. 31, 2008.

(51) Int. Cl. A61K 33/42

(52) U.S. Cl. CPC A61K 33/42
USPC 424/400; 424/604

(57) ABSTRACT

A system for delivering nano-metallic alloys to infected cells in a patient is disclosed. The nano-metallic alloy may be formed from binary, triple, or quadruple elemental compositions complexed in predetermined percentages of monosodium phosphate monohydrate and disodium phosphate heptahydrate. The nano-metallic alloy may be capable of eliminating infectious microorganisms within infect cells or legions without harming the cells or tissues. The system may also include a method of administering a predetermined concentration of the nano-metallic alloy in the complexing solution in the vicinity of the infected cells of legions to kill the foreign matter.

### CROSS-REFERENCE TO RELATED APPLICATION

This is a continuation of U.S. patent application Ser. No. 13/299,791, filed Nov. 18, 2011, which is a divisional of U.S. patent application Ser. No. 12/512,726, filed Jul. 30, 2009, now abandoned, which claims the benefit of U.S. Provisional Patent Application No. 61/085,375, filed Jul. 31, 2008.

### FIELD OF THE INVENTION

This invention is directed to a system and method of treatment of viral and bacterial infections, and more particularly, to a system and method of treatment of viral or bacterial infections with site specific delivery of metallic alloys.

### BACKGROUND OF THE INVENTION

Bacterial resistance to conventional antibiotics is an alarming health hazard. Several suggestions to control the amount of antibiotics by either novel delivery systems such as chewable tablets or localized delivery of the antimicrobials have been described in the prior art, such as in Published U.S. Patent Application No. 2008/0160067 to Boeckh et al., which describes a chewable tablet loaded with a cocktail of antibiotics.

Silver based antimicrobials in solid and ionic forms have also been described in the prior art to be effective against bacterial infection. However, the extent of what is disclosed in the prior art describes either different compositions of sliver/sliver ions, encapsulation or impregnation for external use, particularly as a disinfectant on the body surface. For example, Published U.S. Patent Application No. 2008/0181951 to Holladay et al., describes the efficacy of silver particles formed from elemental silver coated with ionic silver for external treatment of bacterial manifestation at the exterior surfaces.

Chronic osteomyelitis is an intractable inflammation of the bone caused by pathogenic bacteria and is associated with the destruction of bone tissues and vascular channels. The inflammation is characterized by a predominant presences of leukocytes and macrophages, which contribute to the destruction of bone tissues. Staphylococcus aureus is one causative agent of osteomyelitis. S. Aureus can grow in a temperature range of between about 15 and 45 degrees Fahrenheit.

Salmonella is another cause of osteomyelitis, especially in those with sickle cell or other diseases that weaken the immune system. This bacteria is gram negative and rod shaped. The bacteria is resistant to ampicillin, streptomycin, kanamycin, chloramphenicol, tetracycline and sulfonamides.

### SUMMARY OF THE INVENTION

This invention is directed to a system for delivering nano-metallic alloys to infected cells in a patient is disclosed. The nano-metallic alloy may be formed from binary, triple, or quadruple elemental compositions complexed in predetermined percentages of monosodium phosphate monohydrate and disodium phosphate heptahydrate. The nano-metallic alloy may be capable of eliminating infectious microorganisms within infect cells or legions without harming the cells or tissues. The system may also include a method of placement of a predetermined concentration of the nano-metallic alloy in the complexing solution in the vicinity of the infected cells of legions to kill the foreign matter.

The system is configured to deliver nano-metallic alloys to infected cells in a patient. The nano-metallic alloy may be formed from a base formed from copper that is complexed with Ag, Li, Zn or Mn in a monosodium phosphate monohydrate and disodium phosphate heptahydrate. In one embodiment, the material may be complexed with one element selected from the group consisting of: Co, Ag, Zn, Li, Mn, Gd, Ho, Ce, and Sm. The element may be used in concentrations between about one percent and about 99 percent. In another embodiment, the nano-metallic alloy may be formed from 20-30 percent copper and 70-80 percent of one of the group consisting of: Ag, Zn and Mn. In another embodiment, the nano-metallic alloy may be formed from 30 percent copper and 70 percent of one of the group consisting of: Ag, Zn, Li and Mn.

In another embodiment in which a triple complex is formed, the material may be complexed with one alloy selected from the group consisting of: Ag—Zn, Ag—Li, Ag—Co, Ag—Ce, Ag—Ho, Ag—Sm, Zn—Co, Zn—Gd, Zn—Ho, Zn—Mn, and Zn—Ce. The alloy may be used in concentrations between about one percent and about 99 percent. For example, the nano-metallic alloy may be formed from 20-30 percent copper and 70-80 percent of one of the following: Ag—Zn, Ag—Li, Ag—Co, Ag—Ce, Ag—Ho, Ag—Sm, or Ag—Mn. The nano-metallic alloy may be formed from 30 percent copper and 70 percent Ag—Zn of one of the following: Ag—Zn, Ag—Li, Ag—Co, Ag—Ce, Ag—Ho, Ag—Sm, or Ag—Mn. The Ag may be 80-90 percent of one of the following: Ag—Zn, Ag—Li, Ag—Co, Ag—Ce, Ag—Ho, Ag—Sm, or Ag—Mn complex. In other embodiments, the amount of Zn may be 80-90 percent of the alloys having one of the following consisting of Ag, Cu, Co, Ce, Sm, Ho, Li and Mn.

In another embodiment in which a quadruple complex is formed, the material may be complexed with one alloy selected from the group consisting of: Ag—Zn—Li, Ag—Zn—Co, Zn—Co—Ho, Zn—Co—Ce, Zn—Gd—Mn, and Zn—Co—Gd. The alloy may be used in concentrations between about one percent and about 99 percent. The nano-metallic alloy may be formed from 20-30 percent cooper and 70-80 percent of one of the alloys listed. A ratio of Ag—Zn—Li may be about 80:10:10. The nano-metallic alloy may be less than 300 nanometers in size. In one embodiment, the nano-metallic alloy may be between one nanometer and 100 nanometers in size.

An advantage of this invention is that the nano-metallic alloy may be capable of eliminating infectious microorganisms within infect cells or legions without harming the cells or tissues.

These and other embodiments are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a monograph of normal osteoblasts.

FIG. 2 is a monograph of infected osteoblasts.

FIG. 3 is a graph of use of different concentrations of nano-metallic alloys on infected cells.

FIG. 4 is a graph of the effectiveness of exposing one nanogram per milliliter of a nano-metallic alloy to infected cells over a long period of time.

FIG. 5 is a graph of a MTT assay for the osteoblast cells treated with nanoparticles for two hours to determine the toxicity of the nano-metallic alloys.

FIG. 6A is a display of osteoblasts that were not infected nor treated with nanoparticles.

FIG. 6B is a display of osteoblast cells that were treated with bacteria without any nanoparticle treatment, and the green spots represent S. Aureus infection.

FIG. 6C is a display of infected osteoblast cells that were treated with gentamycine and shows that there are no significant numbers of dead bacteria, which is displayed with a red color.

FIG. 6D is a display of cells that were infected with S. Aureus and treated with nanoparticles and gentamycine. The red spots displayed represent significant killing of intracellular bacteria.

FIG. 7 is a confocal image of bone cells that have been treated with antibiotics and include living bacteria.

FIG. 8 is a confocal image of bone cells that have been treated with antibiotics and with gentamycine and include living bacteria.

FIG. 9 is a confocal image of bone cells that have been treated with nano-particles without antibiotics and that shows substantial numbers of dead bacteria, which provides evidence that nanoparticles work efficiently without adding antibiotics.

### DETAILED DESCRIPTION OF THE INVENTION

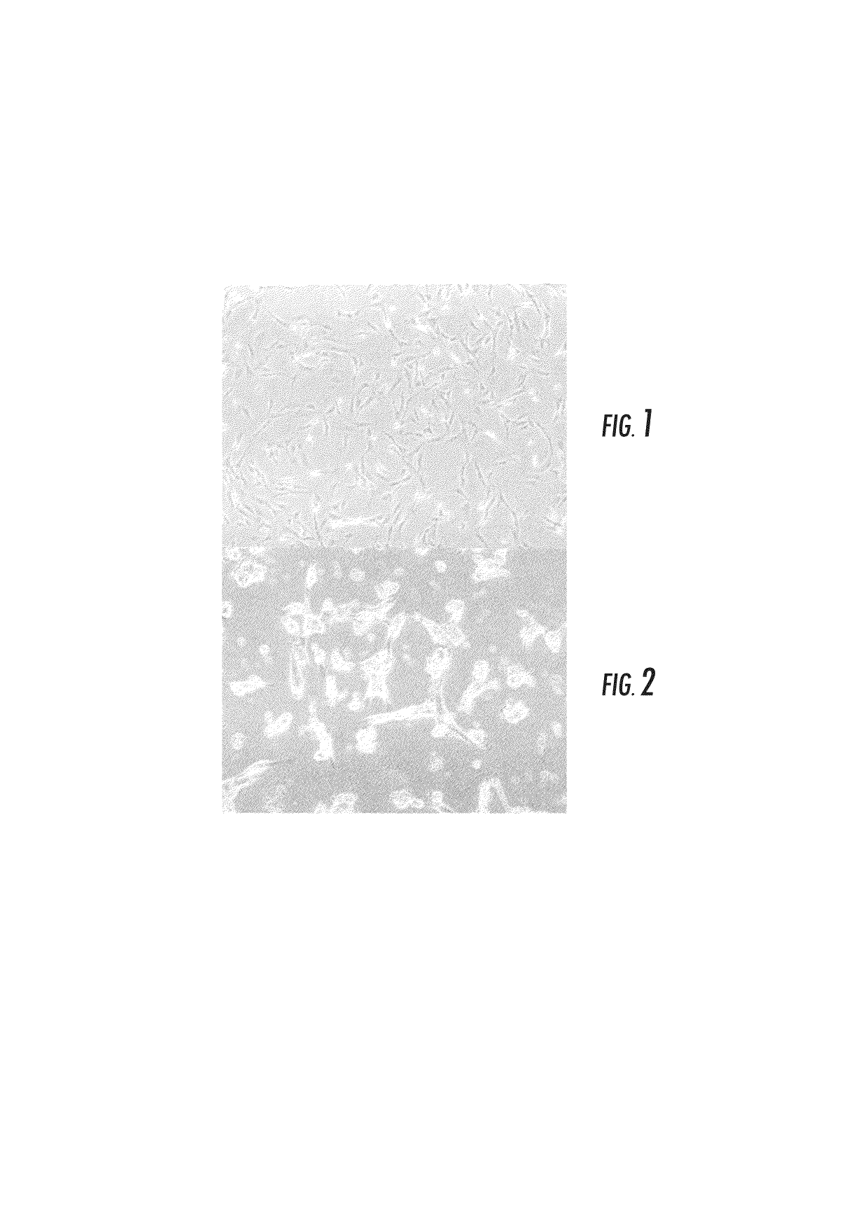

As shown in FIGS. 1-9, the invention is directed to a system for delivering nano-metallic alloys to infected cells in a patient. The nano-metallic alloy may be formed from binary, triple, or quadruple elemental compositions complexed in predetermined percentages of monosodium phosphate monohydrate and disodium phosphate heptahydrate. The nano-metallic alloy may be capable of eliminating infectious microorganisms within infect cells or legions without harming the cells or tissues. The system also includes a method of placement of a predetermined concentration of the nano-metallic alloy in the complexing solution in the vicinity of the infected cells of legions to kill the foreign matter.

The nano-metallic alloy may be formed from a base formed from copper that is complexed with monosodium phosphate monohydrate and disodium phosphate heptahydrate. The nano-metallic alloy may formed from a binary complex such that it has copper as the base complexed in a predetermined percentage with monosodium phosphate monohydrate and disodium phosphate heptahydrate. The nano-metallic alloy may also be complexed with one element, such as, but not limited to: Co, Ag, Zn, Li, Mn, Gd, Ho, Ce, or Sm. The element may be used in concentrations between about one percent and about 99 percent. In another embodiment, the complexed nano-metallic alloy is formed from about 20-30 percent Cu and about 70-80 percent Ag, Zn or Mn. In yet another embodiment, the complexed nano-metallic alloy may be formed from about 30 percent Cu and about 70 percent Ag, Zn, Li or Mn.

In an alternative embodiment, the nano-metallic alloy may formed from a triple complex such that it has copper as the base complexed in a predetermined percentage with monosodium phosphate monohydrate and disodium phosphate heptahydrate. The nano-metallic alloy may also be complexed with an alloy, such as, but not limited to: Ag—Zn, Ag—Li, Ag—Co, Ag—Ce, Ag—Ho, Ag—Sm, Ag—Mn, Zn—Co, Zn—Gd, Zn—Ho, Zn—Mn, and Zn—Ce. The element may be used in concentrations between about one percent and about 99 percent. In another embodiment, the triple complexed nano-metallic alloy may be formed from 20-30 percent Cu and 70-80 percent Ag—Zn, such that the amount of Ag is at least two to three times the amount of Zn. In another embodiment, the triple complexed nano-metallic alloy may be formed from 20-30 percent Cu and 70-80 Ag—Zn, such that the Ag constitutes 80-90 percent of the Ag—Zn complex. The formulation of this triple complexed nano-metallic alloy may be applicable to all Ag complexes disclosed herein. In yet another embodiment, the triple complexed nano-metallic alloy may be formed from 20-30 percent Cu and 70-80 percent Zn—X alloy, where X refers to all elements referred above in this paragraph, such that Zn composes 80-90 percent of the composition between Zn—X.

In another alternative embodiment, the nano-metallic alloy may formed from a quadruple complex such that it has copper as the base complexed in a predetermined percentage with monosodium phosphate monohydrate and disodium phosphate heptahydrate. The nano-metallic alloy may also be complexed with an alloy, such as, but not limited to: Ag—Zn—Li, Ag—Zn—Co, Zn—Co—Ho, Zn—Co—Ce, Zn—Gd—Mn, and Zn—Co—Gd. The element may be used in concentrations between about one percent and about 99 percent. In another embodiment, the quadruple complexed nano-metallic alloy may be formed from 20-30 percent Cu and 70-80 percent Ag—Zn—Li where the ratio of Ag:Zn:Li is about 80:10:10. Similar fractions are applicable with the triple complexes such that the leading element fraction is about 70-80 percent and the remaining two elements vary from about 10-20 percent.

The nano-metallic alloy may have any appropriate size. In at least one embodiment, the nano-metallic alloy may be less than 300 nanometers in size. In another embodiment, the nano-metallic alloy may be between one nanometer and 100 nanometers in size.

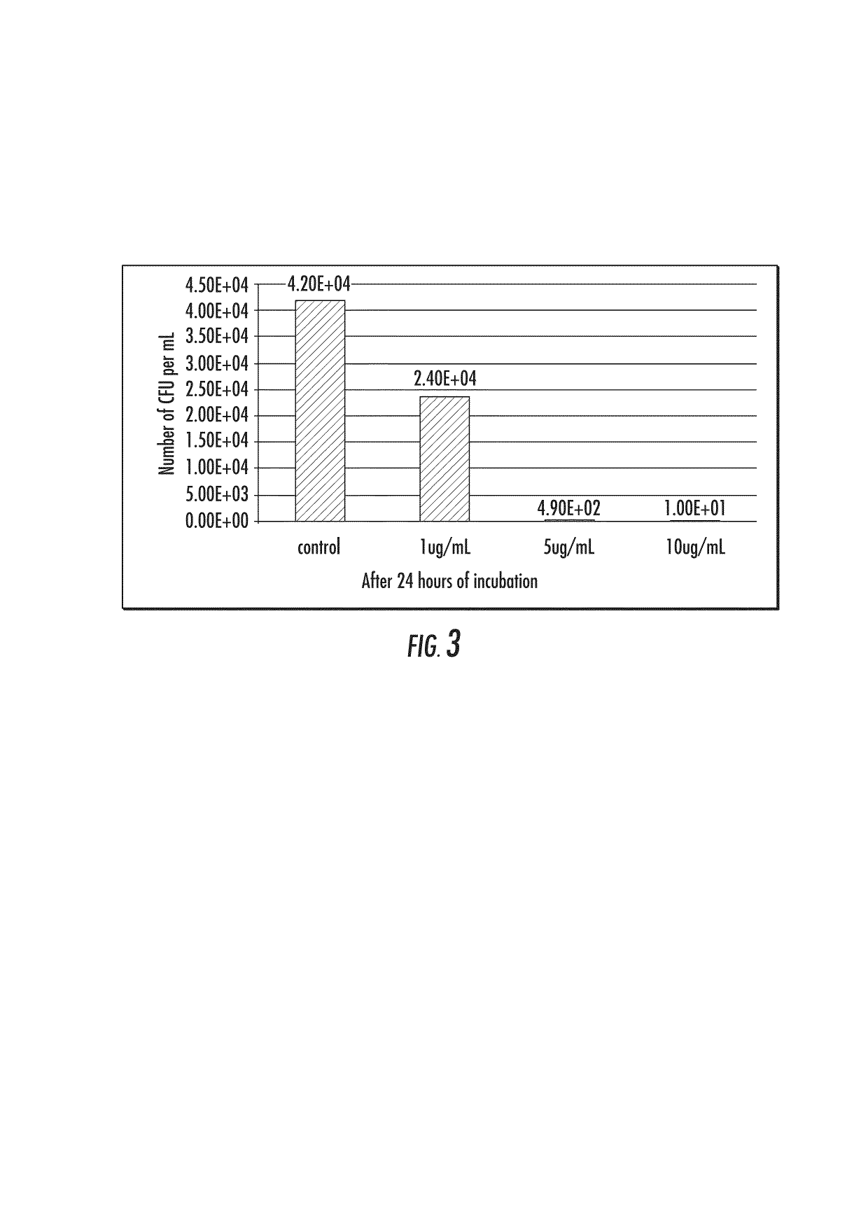

The system 10 for delivering nano-metallic alloys to infected cells in a patient has been used to kill infected cells. In particular, bone cells were first incubated with S. Aureus in order to induce infection in the osteoblasts. FIG. 1 shows a monograph of normal osteoblasts, and FIG. 2 shows a monograph of infected osteoblasts. Gentimycine, which is an antibiotic that does not penetrate osteoblasts, was added to clear the extracellular S. Aureus. Different concentrations of the nano-metallic alloys were added to the infected cells and allowed to incubate for different times. The osteoblasts were then lysed, and the intracellular content was plated to determine the effectiveness of the nano-metallic alloy-mixture. FIG. 3 shows the effectiveness of the different concentrations of the nano-metallic alloy. The results displayed correlate with 24 hours of incubation. As the results clearly demonstrate, as little as one micrograms per milliliter of the nano-metallic mixture was able to substantially reduce the internal infections of the bone cells. In addition, use of 10 micrograms per milliliter of the nano-metallic alloy caused almost a total elimination of the infection.

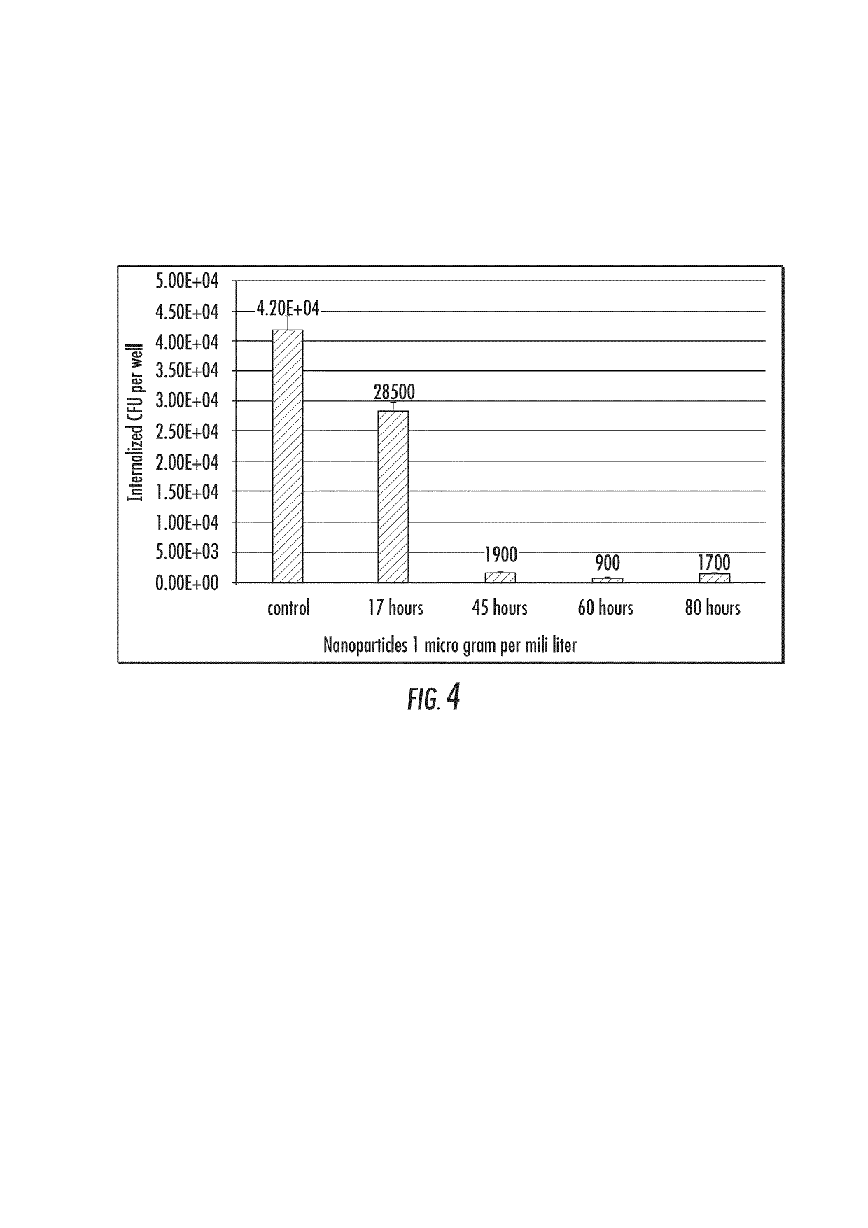

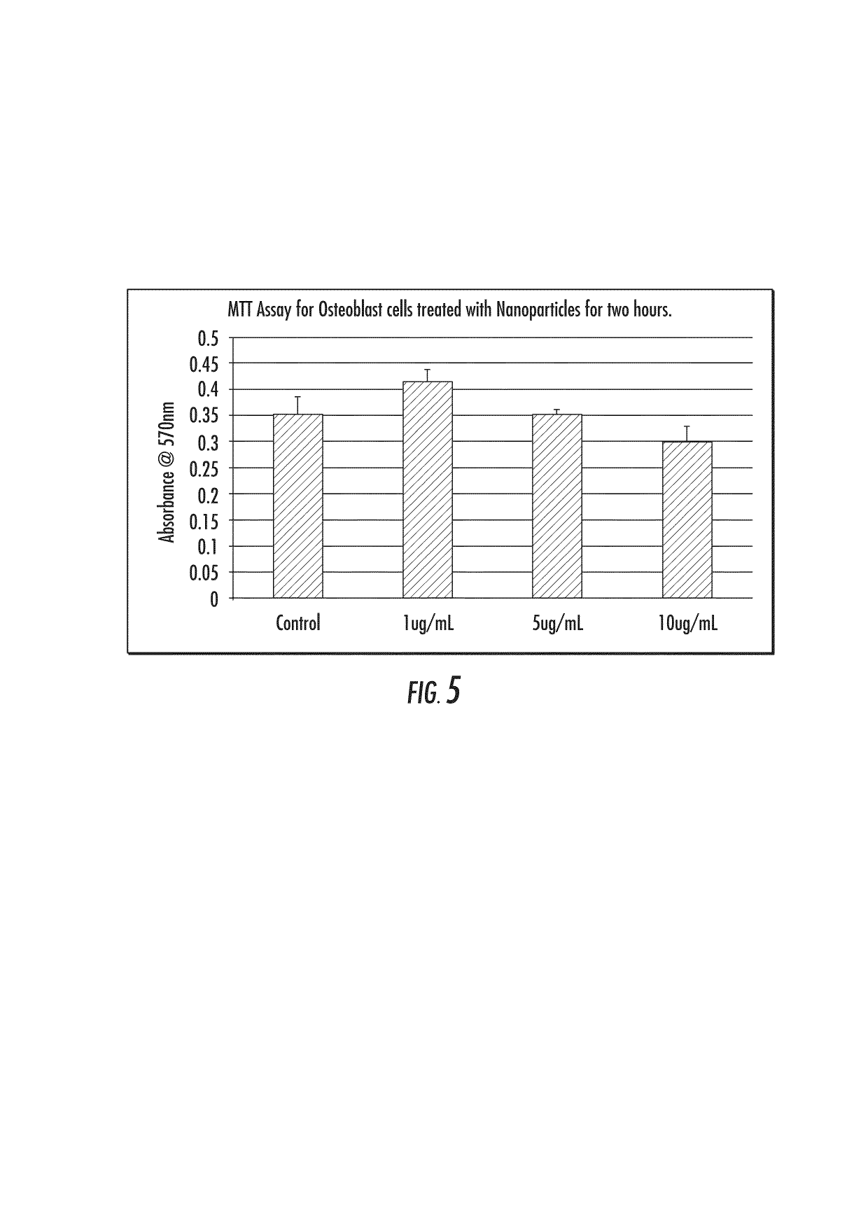

The effectiveness and the durability of the treatment over a long period of time is demonstrated in the data shown in FIG. 4. The results correlated to use of one microgram pre milliliter of nano-metallic alloy incubated for a time period up to 80 hours. As is clearly demonstrated, one micro gram per milliliter was effective and capable of reducing the infection substantially for a long time. The toxic implication of the nano-metallic alloy on the osteoblasts was tested and is shown in FIG. 5. There was no toxic effect on the osteoblasts for up to 10 micrograms per milliliter of the nano-metallic mixture.

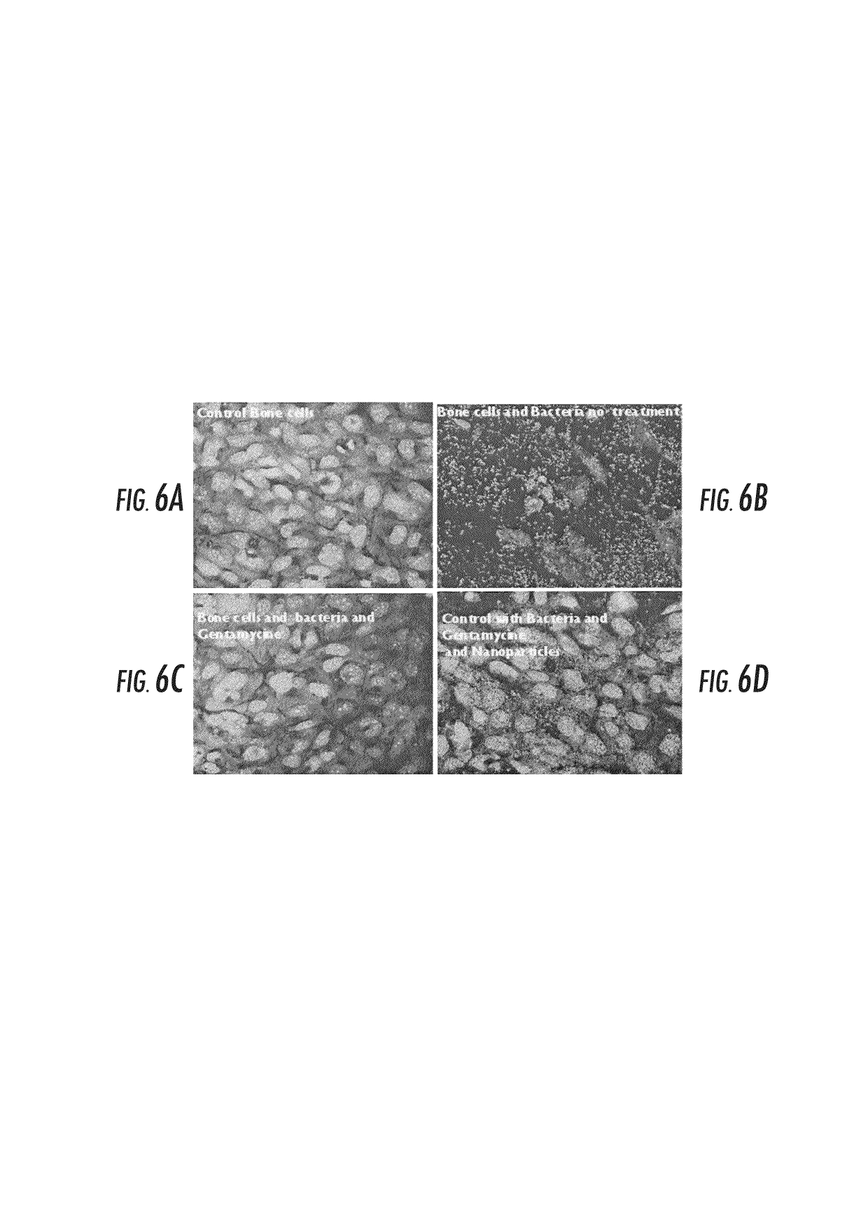

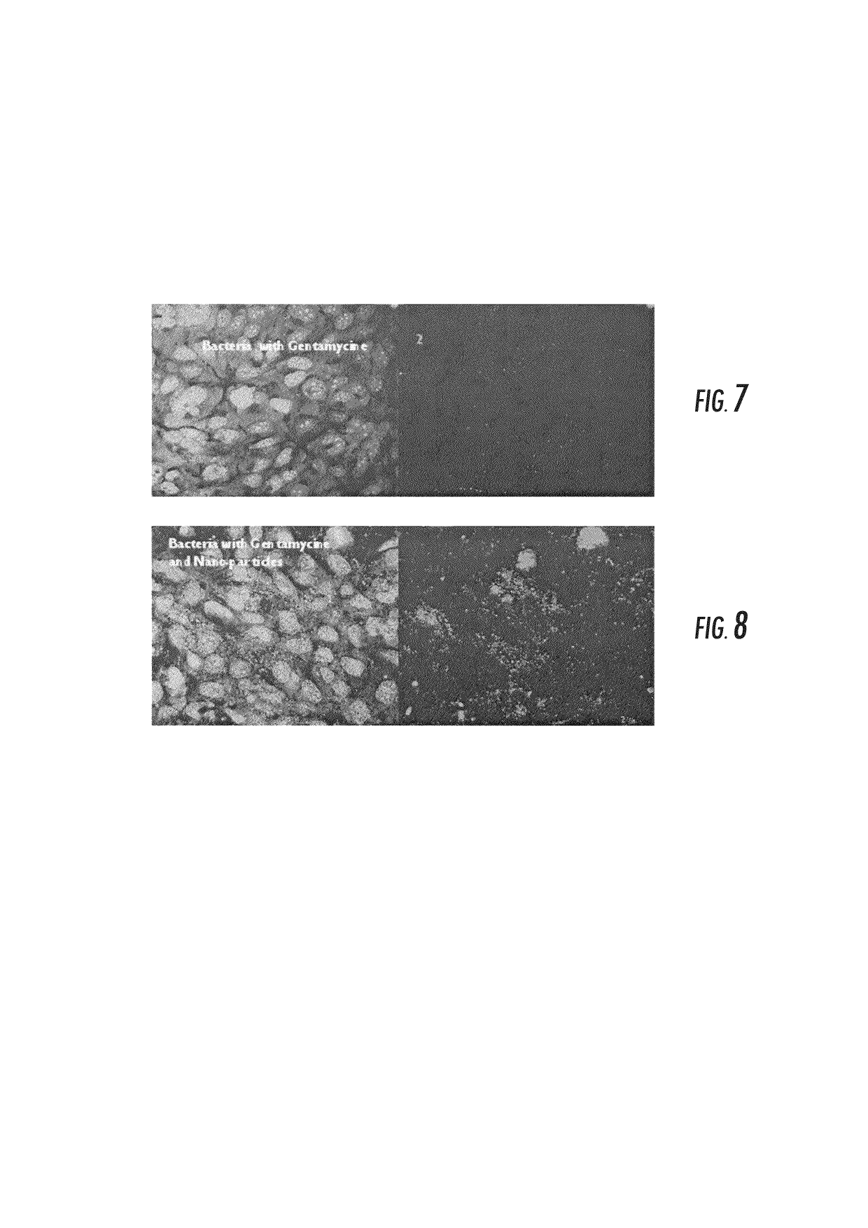

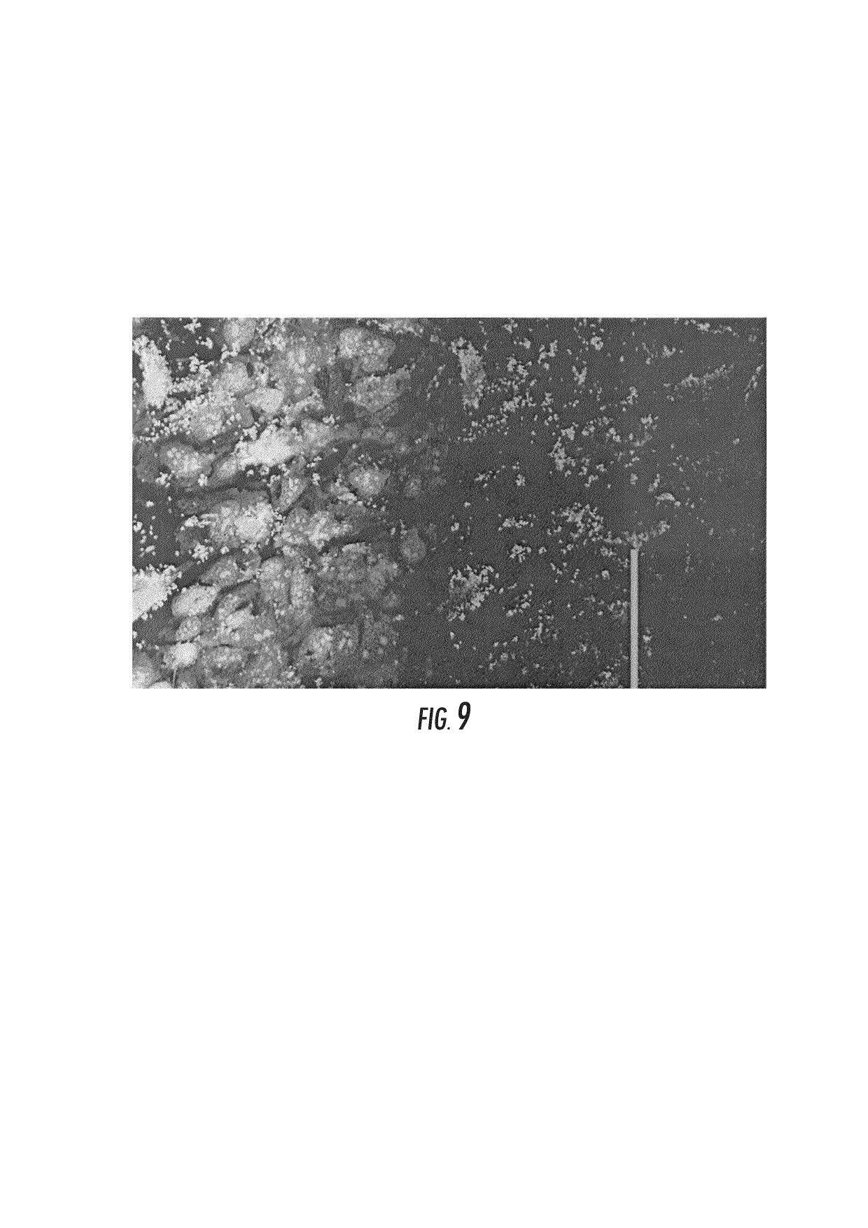

Confocal microscopy provided the evidence for intracellular bacteria killing. Osteoblast cells were cultured on 18 mm cover slips in a six well plate. Cells were treated with nanoparticles 1 ug/mL. The stain used was from invitrogen (L-7002) with two types of Dyes, SYTO 9 dye, 3,34 mM (Component A), 300 μL solution in DMSO and Propidium iodide PI, 20 mM (Component B), 300 μL solution in DMSO. The plates were incubated in the dark for 15 minutes after which the coverslips with growing co-cultured cells were gently removed and were placed on a glass slide containing 50 μL of 10%A glycerol with cells lying between the cover slip and the glass slide. The slide was observed under a confocal microscope using bandpass filters for red and green color. Images were obtained at different working distances and were superimposed by an Olympus CCD camera software to get images with dual color. FIG. 6A displays osteoblasts that were not infected nor treated with nanoparticles. FIG. 6B displays osteoblast cells that were treated with bacteria without any nanoparticle treatment, and the green spots represent S. Aureus infection. FIG. 6C displays infected osteoblast cells that were treated with gentamycine. Gentamycine is an antibiotic that is not capable of penetrating the bone cells membrane. FIG. 6C shows that there are no significant numbers of dead bacteria, which is displayed with a red color. FIG. 6D displays cells that were infected with S. Aureus and treated with nanoparticles and gentamycine. The red spots displayed represent significant killing of intracellular bacteria. FIG. 7 displays a confocal image of bone cells that have been treated with antibiotics and include living bacteria. FIG. 8 displays a confocal image of bone cells that have been treated with antibiotics and with gentamycine and include living bacteria.

FIG. 9 displays a confocal image of bone cells that have been treated with nano-particles without antibiotics. FIG. 9 shows substantial numbers of dead bacteria, which provides evidence that nanoparticles work efficiently without adding antibiotics. In addition, the nanoparticles killed both intracellular and extracellular bacteria.

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A method of reducing infected cells in a patient, comprising:
administering a nano-metallic alloy formed from a base that is formed from copper complexed with Ag, Li, Zn or Mn in a monosodium phosphate monohydrate and disodium phosphate heptahydrate;
wherein the nano-metallic alloy is administered at a concentration of at least one microgram per milliliter.

2. The method of claim 1, wherein administering the nano-metallic alloy comprises administering the nano-metallic alloy complexed with one element selected from the group consisting of: Co, Ag, Zn, Li, Mn, Gd, Ho, Ce, and Sm.

3. The method of claim 2, wherein administering the nano-metallic alloy comprises administering the nano-metallic alloy complexed with one element used in concentrations between about one percent and about 99 percent.

4. The method of claim 2, wherein administering the nano-metallic alloy comprises administering the nano-metallic alloy formed from 20-30 percent copper and 70-80 percent of one of the group consisting of: Ag, Zn, Li and Mn.

5. The method of claim 4, wherein administering the nano-metallic alloy comprises administering the nano-metallic alloy formed from 30 percent copper and 70 percent of one of the group consisting of: Ag, Zn and Mn.

6. The method of claim 1, wherein administering the nano-metallic alloy comprises administering the nano-metallic alloy complexed with one alloy selected from the group consisting of: Ag—Zn, Ag—Li, Ag—Co, Ag—Ce, Ag—Ho, Ag—Sm, Ag—Mn, Zn—Co, Zn—Gd, Zn—Ho, Zn—Mn, and Zn—Ce.

7. The method of claim 6, wherein administering the nano-metallic alloy comprises administering the nano-metallic alloy used in concentrations between about one percent and about 99 percent.

8. The method of claim 7, wherein administering the nano-metallic alloy comprises administering the nano-metallic alloy is formed from 20-30 percent copper and 70-80 percent of one of the group consisting of Ag—Zn, Ag—Li, Ag—Co, Ag—Ce, Ag—Ho, Ag—Sm, and Ag—Mn.

9. The method of claim 8, wherein administering the nano-metallic alloy comprises administering the nano-metallic alloy formed from 30 percent copper and 70 percent of one of the group consisting of Ag—Zn, Ag—Li, Ag—Co, Ag—Ce, Ag—Ho, Ag—Sm, and Ag—Mn.

10. The method of claim 9, wherein administering the nano-metallic alloy comprises administering the nano-metallic alloy formed from Ag being 80-90 percent of one of the group consisting of the Ag—Zn, Ag—Li, Ag—Co, Ag—Ce, Ag—Ho, Ag—Sm, and Ag—Mn complex.

11. The method of claim 6, wherein administering the nano-metallic alloy comprises administering the nano-metallic alloy formed from Zn being 80-90 percent of the alloys having one of the following consisting of Ag, Cu, Co, Ce, Sm, Ho, Li and Mn.

12. The method of claim 1, wherein administering the nano-metallic alloy comprises administering the nano-metallic alloy complexed with one alloy selected from the group consisting of: Ag—Zn—Li, Ag—Zn—Co, Zn—Co—Ho, Zn—Co—Ce, Zn—Gd—Mn, and Zn—Co—Gd.

13. The method of claim 12, wherein administering the nano-metallic alloy comprises administering the nano-metallic alloy used in concentrations between about one percent and about 99 percent.

14. The method of claim 12, wherein administering the nano-metallic alloy comprises administering the nano-metallic alloy formed from 20-30 percent cooper and 70-80 percent of one of the alloys listed.

15. The method of claim 14, wherein administering the nano-metallic alloy comprises administering the nano-metallic alloy having a ratio of Ag—Zn—Li that is about 80:10:10.

16. The method of claim 1, wherein administering the nano-metallic alloy comprises administering the nano-metallic alloy that is less than 300 nanometers in size.

17. The method of claim 1, wherein administering the nano-metallic alloy comprises administering the nano-metallic alloy that is between one nanometer and 100 nanometers in size.

18. A method of reducing infected cells in a patient, comprising:
administering a nano-metallic alloy formed from a base that is formed from copper complexed with Ag, Li, Zn or Mn in a monosodium phosphate monohydrate and disodium phosphate heptahydrate;
wherein the nano-metallic alloy is administered at a concentration of at least ten micrograms per milliliter.

19. The method of claim 18, wherein administering the nano-metallic alloy comprises administering the nano-metallic alloy complexed with one element selected from the group consisting of: Co, Ag, Zn, Li, Mn, Gd, Ho, Ce, and Sm.

20. The method of claim 18, wherein administering the nano-metallic alloy comprises administering the nano-metallic alloy complexed with one alloy selected from the group consisting of: Ag—Zn, Ag—Li, Ag—Co, Ag—Ce, Ag—Ho, Ag—Sm, Ag—Mn, Zn—Co, Zn—Gd, Zn—Ho, Zn—Mn, and Zn—Ce.
